(19) United States
(12) Patent Application Publication
(10) Pub. No.: US 2026/0102185 A1
(43) Pub. Date: Apr. 16, 2026

(54) STRAP FIXING DEVICE

(71) Applicant: Jungu Biomedical Device Co., Ltd, Taipei City (TW)

(72) Inventors: YI-CHEN LIN, Taipei City (TW); WEI-BIN HSU, Taipei City (TW)

(73) Assignee: Jungu Biomedical Device Co., Ltd, Taipei City (TW)

(21) Appl. No.: 19/026,635

(22) Filed: Jan. 17, 2025

(30) Foreign Application Priority Data
Oct. 14, 2024 (TW) 113138969

(51) Int. Cl. A61B 17/70

(52) U.S. Cl. CPC A61B 17/7032

(57) ABSTRACT

A strap fixing device is suitable to fix a strap to a fixed object and includes a body, a fixing piece and a locking piece. The body includes a crown part, a body part and a holding part, wherein the crown part forms a locking hole; the body part is connected to the crown part and forms a fixing hole. The fixing piece is suitable to be penetratingly disposed at the fixing hole and includes a fixing surface and an abutted surface, wherein the fixing surface corresponds to at least a part of a surface of the fixed object, and the abutted surface is configured relative to the fixing surface. The locking piece is suitable to be penetratingly disposed at the locking hole and includes an abutting surface, and the abutting surface optionally abuts against the abutted surface.

### CROSS-REFERENCE TO RELATED APPLICATION

This non-provisional application claims priority under 35 U.S.C. § 119(a) on Patent Application No(s).113138969 filed in Taiwan, R.O.C. on Oct. 14, 2024, the entire contents of which are hereby incorporated by reference.

### BACKGROUND OF THE INVENTION

### Field of the Invention

The present disclosure provides a strap fixing device, and in particular with respect to a strap fixing device that can fix a strap to a fixed object in a narrow operating space.

### Description of the Related Art

Spinal orthopedics is a very important discipline in medical care. For example, spinal orthopedic diseases such as degenerative lesions of the spine, herniation of intervertebral disc, sciatica, scoliosis and bone spur formation have a profound impact on the national health, and because modern people tend to maintain the same sedentary and standing posture for a long time in their lives, these diseases have become one of the national diseases that plague the people who live in this country.

Usually, the main treatment for the above-mentioned diseases is open surgery and minimally invasive surgery. Open surgery involves using a scalpel to cut through the musculature near the spine and placing implants around the spine that can share the load or stabilize tissue during activities of daily living, such as bone nails or bone plates. Although open surgery is not a complicated procedure for experienced surgeons, patients need a longer recovery time after surgery. In contrast, minimally invasive surgery involves an incision about the size of a coin in the epidermis close to the spine and places the implant into the patient’s body through a special implanting tool, which not only has less bleeding, but also has a shorter recovery time after surgery, and the patient can get out of bed the next day after surgery and be discharged within a week, the above-mentioned advantage makes minimally invasive surgery be an option for many patients.

### BRIEF SUMMARY OF THE INVENTION

However, in the past, when fixing the spine strap around the spine, medical staff needed to open an incision on both sides of the patient’s spine, and the strap passes through the soft tissue such as ligament or cartilage on the spine by an implanting tool from the incision at one side, and then the strap is clamped and fixed by a clamping tool from the incision at the other side, not only does it take a lot of time to find the strap that is penetratingly disposed to the other side, but the increase in incisions also brings additional burden on patients.

The inventor devoted himself to careful research and developed a strap fixing device that can fix a strap to a fixed object in a narrow operating space, in order to achieve the effect of reducing the incision and shortening the operation time.

The present disclosure provides a strap fixing device, suitable to fix a strap to a fixed object and including: a body, a fixing piece and a locking piece. The body includes a crown part, a body part and a holding part, wherein the crown part forms a locking hole, and the locking hole extends along a longitudinal direction; the body part is connected to the crown part and forms a fixing hole, the fixing hole extends along a transverse direction and communicates the locking hole; the holding part is connected to the body part and forms a holding groove and a through hole, the holding groove is suitable for clamping with at least a part of the fixed object, and the holding groove communicates to the fixing hole through the through hole. The fixing piece is suitable to be penetratingly disposed at the fixing hole and includes a fixing surface and an abutted surface, wherein the fixing surface corresponds to at least a part of a surface of the fixed object, and the abutted surface is configured relative to the fixing surface. The locking piece is suitable to be penetratingly disposed at the locking hole and includes an abutting surface, and the abutting surface optionally abuts against the abutted surface.

In one embodiment of the disclosure, the locking hole is a screw hole, and the locking piece is suitable to be screwed into the screw hole.

In one embodiment of the disclosure, the body part includes at least one bearing surface, and the bearing surface is configured to be adjacent to the fixing hole. The fixing piece further includes at least one clamping surface, the clamping surface is configured to be adjacent to the fixing surface and suitable to abut against the bearing surface.

In one embodiment of the disclosure, the bearing surface and the clamping surface are plural, these bearing surfaces are symmetrically configured relative to the through hole, and these clamping surfaces are symmetrically configured relative to the fixing surface.

In one embodiment of the disclosure, the fixed object is a cylinder, and a cross-section of a groove wall of the holding groove substantially forms a major arc corresponding to a cross-section of the cylinder.

Accordingly, the strap fixing device of the present disclosure can penetratingly dispose the fixing piece at the fixing hole of the body, and through the abutting surface of the locking piece abutting against the abutted surface of the fixing piece, the effect of fixing the strap on the fixed object is achieved. In addition, the strap can be fixed on one side of the biological tissue through the above-mentioned strap fixing device, so there is no need to increase the incision or operating space, and the effect of reducing the incision and shortening the operation time can be achieved.

In order to make the above-mentioned features and advantages of the present disclosure more obvious and easier to understand, the following embodiments are specially given, together with the drawings are described in detail as follows.

### BRIEF DESCRIPTION OF THE DRAWINGS

FIG. 1 is a schematic perspective view of a strap fixing device fixing a strap to a fixed object according to an embodiment of the present disclosure.

FIG. 2 is a schematic exploded view of the strap fixing device of FIG. 1.

FIG. 3 is a schematic front view of the body in FIG. 2.

FIG. 4 is a schematic front cross-sectional view of FIG. 2.

FIG. 5 is a schematic right-hand cross-sectional view of FIG. 2.

FIG. 6 is a schematic front view of the fixing piece in FIG. 2.

FIG. 7 is a schematic front cross-sectional view of the locking piece in FIG. 2.

FIG. 8 is a schematic perspective view of configuring a fastener through an implanting device.

FIG. 9 is a schematic perspective view of the fastener and the strap penetratingly disposed at the biological tissue.

FIG. 10 is a schematic perspective view of configuring the strap to the fixed object.

FIG. 11 is a schematic cross-sectional view of penetratingly disposing the fixing piece at the body.

FIG. 12 is a schematic cross-sectional view of engaging and abutting the fixing piece with the body.

FIG. 13 is a schematic cross-sectional view of locking the locking piece at the body.

### DETAILED DESCRIPTION OF THE INVENTION

The aforementioned and other technical contents, characteristics and effects of the present disclosure can be clearly presented by the detailed description of preferable embodiments together with the attached drawings. It’s worth mentioning that the direction terms mentioned in the following embodiments, such as: top, bottom, left, right, front or back are only referred to the direction of the drawings. Therefore, the directional terms used are intended to illustrate, not to limit the disclosure. In addition, in the following embodiments, the same or similar elements will use the same or similar reference numerals.

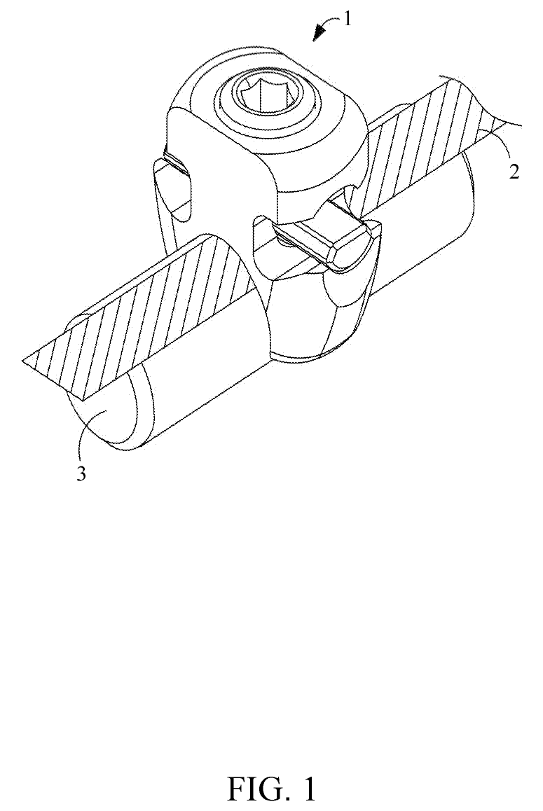

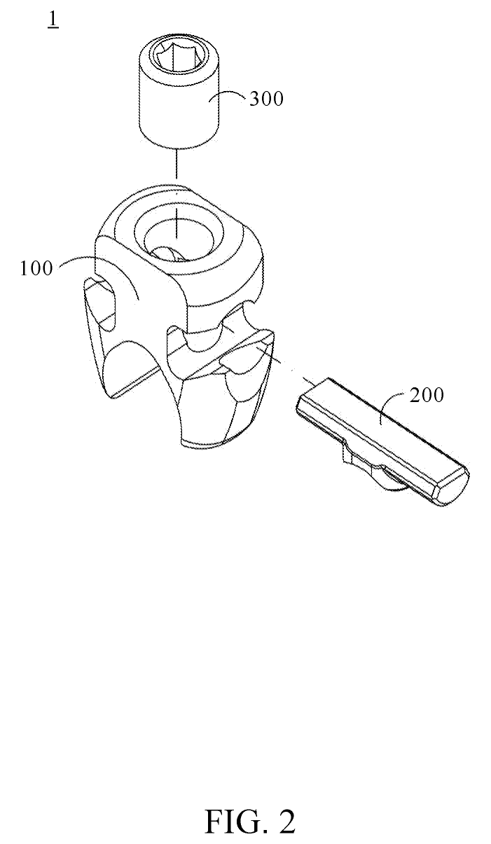

Referring to FIGS. 1 and 2, FIG. 1 is a schematic perspective view of a strap fixing device fixing a strap to a fixed object according to an embodiment of the present disclosure, and FIG. 2 is a schematic exploded view of the strap fixing device of FIG. 1. The strap fixing device 1 of the present embodiment is suitable for fixing a strap 2 to a fixed object 3, wherein the strap 2, for example, is a biocompatible and elastic fastening band, and the fixed object 3, for example, is a cylindrical medical-grade spinal fusion device suitable for implanting into the human body, but the present disclosure is not limited thereto. In other possible embodiments, the strap 2 may also be a common shoelace or suture, and the fixed object 3 may also be a common wooden rod or sewing needle. On the other hand, the strap fixing device 1 includes a body 100, a fixing piece 200 and a locking piece 300, wherein the fixing piece 200 is suitable for penetratingly disposing at the body 100 along a transverse direction (a direction from the upper left to the lower right in FIG. 2), and the locking piece 300 is suitable for penetratingly disposing at the body 100 along a longitudinal direction (the upper and lower directions in FIG. 2) and abuts against the fixing piece 200.

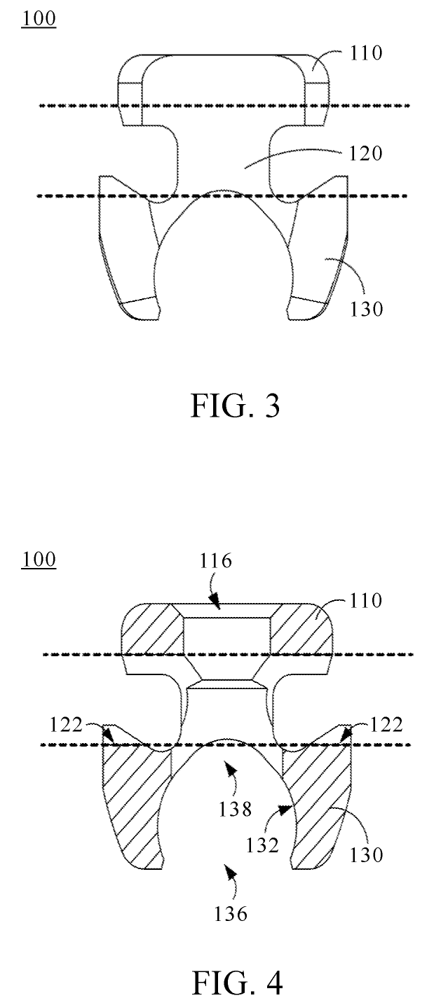

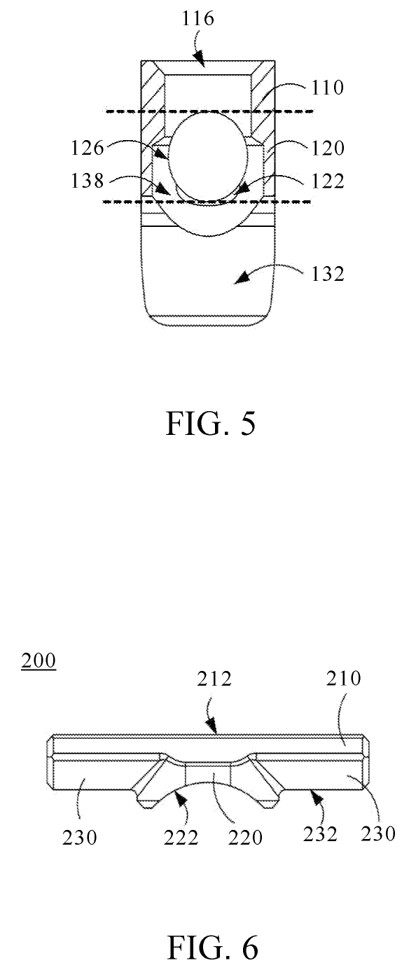

Referring to FIG. 3 to FIG. 5, FIG. 3 is a schematic front view of the body in FIG. 2, FIG. 4 is a schematic front cross-sectional view of FIG. 2, and FIG. 5 is a schematic right-hand cross-sectional view of FIG. 2. In detail, the body 100 may include a crown part 110, a body part 120 and a holding part 130, wherein the crown part 110 is in the shape of a dome with a flat top and forms a locking hole 116. In the present embodiment, the locking hole 116, for example, is a screw hole and extends along a longitudinal direction, but in other possible embodiments, the locking hole 116 may also be a round hole with a smooth inner wall, and the present disclosure is not limited thereto. On the other hand, the body part 120 is connected to the crown part 110 and forms a fixing hole 126, the fixing hole 126 extends along a transverse direction, and the locking hole 116 runs through the entire crown part 110 and communicates the fixing hole 126.

As shown in FIGS. 4 and 5, the holding part 130 is connected to the body part 120 and forms a holding groove 136, and the holding groove 136 is concave along a longitudinal direction and makes the holding part 130 mainly distributed on the left and right sides in FIG. 4. Furthermore, the holding part 130 includes a holding surface 132 facing the bottom side, and the holding surface 132 substantially forms a groove wall of the holding groove 136. In addition, the holding part 130 further forms a through hole 138, in the present embodiment, the through hole 138 for example, extends longitudinally, and the holding groove 136 communicates the fixing hole 126 through the through hole 138. In fact, an upper endpoint and a lower endpoint of the fixing hole 126 can be respectively defined a tangent plane along the transverse direction, the body 100 above the tangent plane at the upper endpoint is defined as the crown part 110, and the body 100 below the tangent plane at the lower endpoint is defined as the holding part 130. The body 100 between the two tangent planes is defined as the body part.

As shown in FIG. 4 and FIG. 5, in some possible embodiments, the body part 120 includes at least one bearing surface 122, and the bearing surface 122 is configured to be adjacent to the fixing hole 126, and is used for bearing the fixing piece 200. In the present embodiment, the number of bearing surfaces 122 is two, and the symmetrical configuration of these two bearing surfaces 122 relative to the through hole 138 is taken as an example, but the present disclosure is not limited thereto.

Referring to FIG. 6, FIG. 6 is a schematic front view of the fixing piece in FIG. 2. In detail, the fixing piece 200 is generally cylindrical and includes an abutted part 210, a fixing part 220 and two clamping parts 230, wherein the abutted part 210 is approximately located at the upper half of the cylinder and includes an abutted surface 212, in the present embodiment, the abutted surface 212 is taken a plane as an example. On the other hand, the fixing part 220 is configured on the bottom side of the abutted part 210 and includes a fixing surface 222, wherein the fixing surface 222 is disposed relative to the abutted surface 212 and corresponds to at least a part of a surface of the fixed object 3. In the present embodiment, the fixing surface 222 is taken a concave arc surface as an example. In addition, the clamping part 230 is approximately the lower half of a cylinder and is symmetrically configured relative to the fixing part 220. Each clamping part 230 includes a clamping surface 232, and the clamping surface 232 is configured relative to the abutted surface 212.

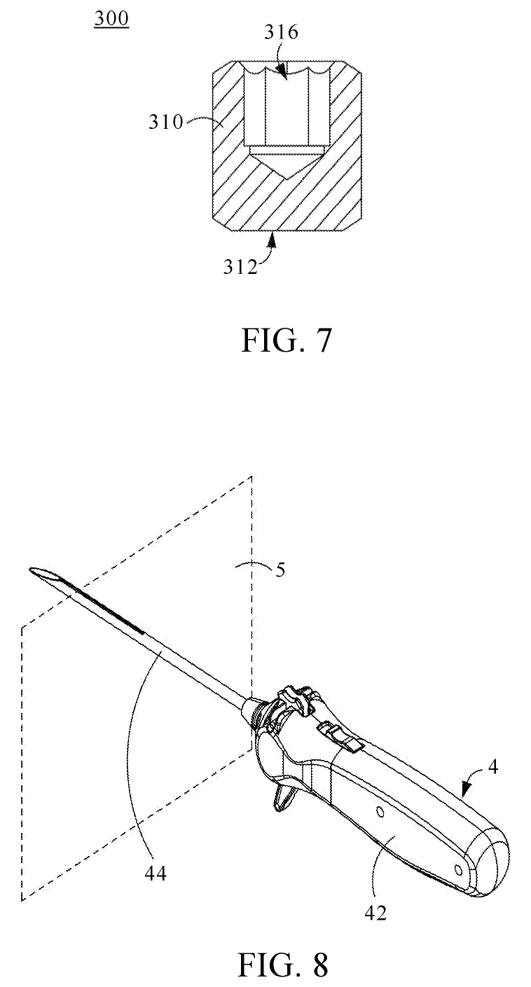

Referring to FIG. 7, FIG. 7 is a schematic front cross-sectional view of the locking piece in FIG. 2. The locking piece 300 of the present embodiment includes a locking body 310, the locking body 310 has an abutting surface 312, and in the present embodiment, the abutting surface 312 is disposed on the bottom side of the locking body 310, and the abutting surface 312 is taken a plane as an example, but the disclosure is not limited thereto. In other possible embodiments, the abutting surface 312 may also be a convex surface, a concave surface, a curved surface or an inclined plane with a slope, as long as it can fully fit with the abutted surface 212 (marked in FIG. 6), all are within the protection scope of the present disclosure. In addition, the locking body 310 is further formed with a mounting hole 316 for a mounting tool to be inserted and penetratingly disposed in the locking hole 116 (marked in FIG. 4).

The following will be described in detail how the strap fixing device of the present embodiment fixes the strap 2 to the fixed object 3. Referring to FIG. 8, FIG. 8 is a schematic perspective view of configuring a fastener through an implanting device. First, an implanting device 4 is provided, the implanting device 4, for example, is a tool used to penetratingly dispose the strap 2 and the fastener used to fix the strap 2 into the biological tissue 5 and includes an operating part 42 and an implanting part 44, wherein the operating part 42 is suitable for the user to hold, and the implanting part 44, for example, is a hollow metal round tube with a spike-shaped end. When the user holds the operating part 42 and abuts the implanting part 44 with the biological tissue 5, since the implanting part 44 is made of metal and has a tiny tip, it can easily penetrate from one side (hereinafter referred to as the proximal side) of the biological tissue 5 to the other side (hereinafter referred to as the distal side) of the biological tissue 5.

It is worth mentioning that, in the present embodiment, the biological tissue 5, for example, is a ligament of an animal, so when the implanting part 44 penetrates it, there is only a hole similar to an outer diameter of the implanting part 44 left at the penetration site without being damaged or fragmented. However, in other possible embodiments, the biological tissue 5 may also be tissues such as epidermis, dermis, cartilage or muscle, and is not necessarily connected to other living tissues during the penetration, that is, the above-mentioned penetrating action and the banding action in the following can be performed in vitro.

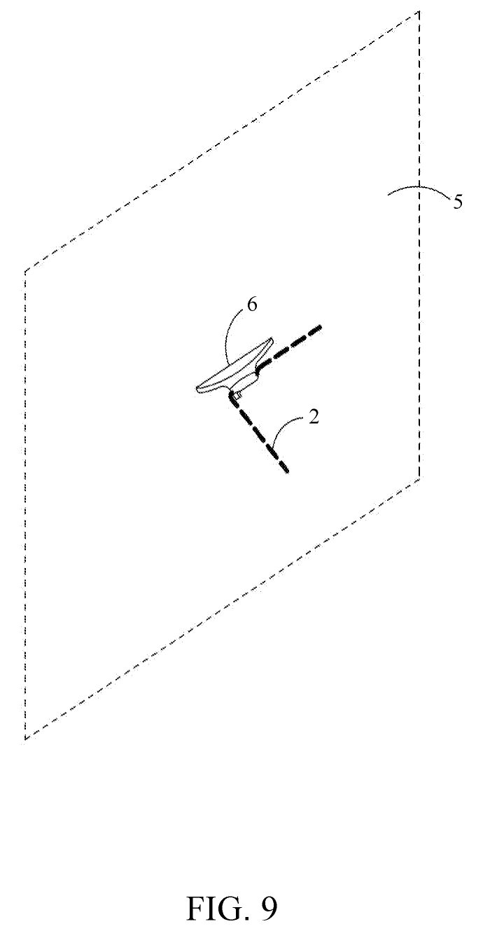

Continuously referring to FIG. 9, FIG. 9 is a schematic perspective view of the fastener and the strap penetratingly disposed at the biological tissue. After the tip of the implanting part 44 passes through the biological tissue 5, the fastener 6 and the strap 2 that are placed inside the implanting part 44 can be pushed forward through a switch of the operating part 44, and the strap 2 has been fastened to the fastener 6 beforehand. In the present embodiment, the fastener 6, for example, is a metal component in the shape of a long strip, and because a length of the fastener 6 is greater than an aperture of the hole left by the implanting part 44 on the biological tissue 5, when the implanting part 44 retreats to the proximal side of the biological tissue 5 along a path of the original penetration, the fastener 6 will be stuck by the biological tissue 5 and stay on the distal side of the biological tissue 5, so that one end of the strap 2 connected to the fastener 6 is also fixed on the distal side of the biological tissue 5. After that, the user can pull the other end of the strap 2 with appropriate force from the proximal side of the biological tissue 5 to ensure that the fastener 6 has been assuredly and stably clamped at the biological tissue 5.

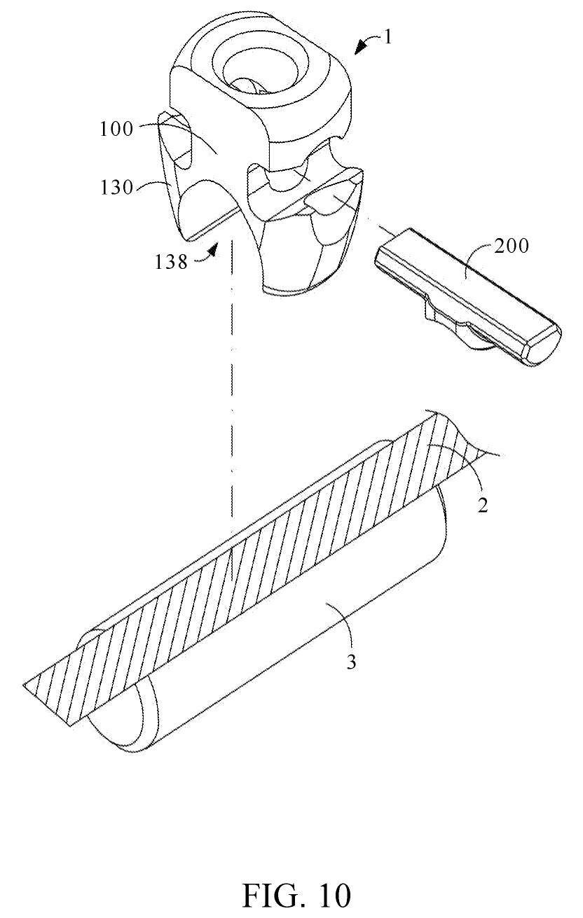

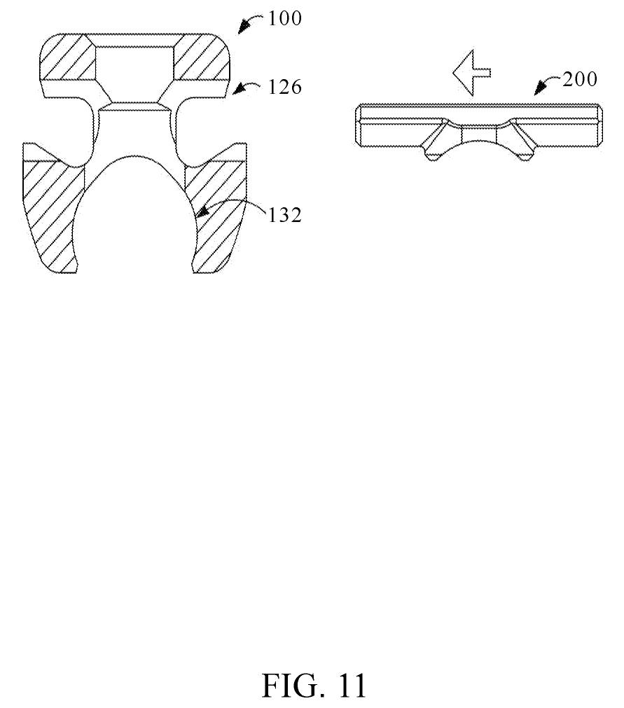

Continued as shown in FIGS. 10 and 11, the user can configure the fixed object 3 at an appropriate position on the proximal side of the biological tissue 5, and temporarily configure the strap 2 on the surface of the fixed object 3. At the same time, the fixing piece 200 can be penetratingly disposed in the fixing hole 126 along the direction of the arrow in FIG. 11, that is, along the transverse direction. Next, the body 100 is longitudinally fastened to the strap 2 and the fixed object 3, so that the strap 2 and the fixed object 3 are embedded in the through hole 138 of the holding part 130.

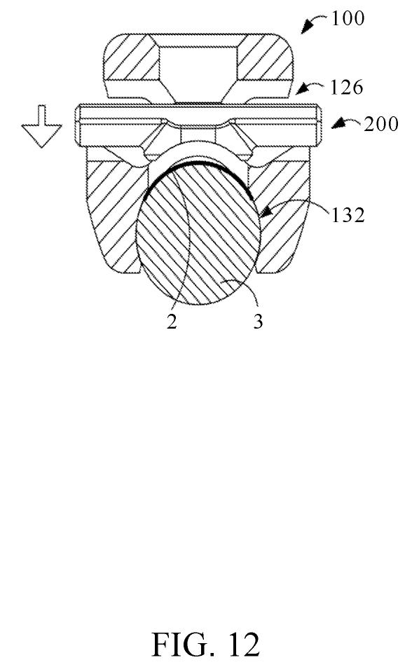

As shown in FIG. 12, because the groove wall of the through hole 138, that is, the cross-section of the holding surface 132 substantially forms a major arc corresponding to the cylindrical cross-section of the fixed object 3, that is, the surface of the holding surface 132 abutting against the fixed object 3 is greater than half. Through such a configuration, it is ensured that the strap 2 is difficult to move with respect to the fixed object 3 along the surface of the fixed object 3.

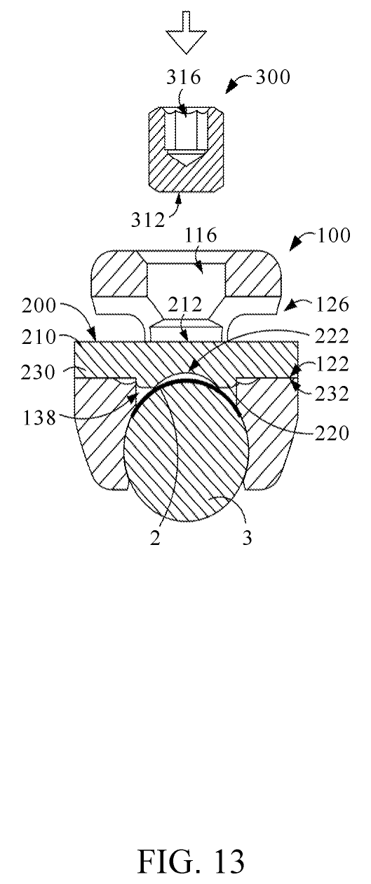

As shown in FIGS. 12 and 13, the fixing piece 200 is continuously pressed down, so that the clamping surfaces 232 of the clamping part 230 at both sides will respectively abut against the bearing surface 122 of the body 100, and the fixing surfaces 222 of the fixing part 220 will pass through the through hole 138 and extend into the holding groove 136. At this time, the user can insert an operating tool such as a screwdriver into the mounting hole 316 of the locking piece 300, so that the locking piece 300 is kept relatively close to the body 100 in a direction of the abutting surface 312 facing downward. In the present embodiment, when the locking piece 300 is completely screwed into the locking hole 116, the abutting surface 312 will abut against the abutted surface 212 of the fixing piece 200, forcing the fixing piece 200 to move downward, and then the strap 2 is fixed to the surface of the fixed object 3 stably through the fixing surface 222. In addition, since the clamping part 230 is symmetrically configured relative to the fixing part 220, when the locking piece 300 is locked downward, the fixing piece 200 can be evenly stressed without deflecting relative to the body 100, thus improving the success rate of fixation.

Accordingly, the user only needs to operate on the proximal side of the biological tissue 5, and one end of the strap 2 can be fixed to the distal side of the biological tissue 5, and the other end can be firmly fixed to the fixed object 3 through the strap fixing device 1, so that the incision that needs to be opened in order to improve the operating space can be effectively reduced, and the time required for operation can also be shortened.

While the present invention has been described by means of preferable embodiments, those skilled in the art should understand the above description is merely embodiments of the invention, and it should not be considered to limit the scope of the invention. It should be noted that all changes and substitutions which come within the meaning and range of equivalency of the embodiments are intended to be embraced in the scope of the invention. Therefore, the scope of the invention is defined by the claims.

What is claimed is:

1. A strap fixing device, suitable to fix a strap to a fixed object, the strap fixing device comprising:
a body, comprising:
a crown part, forming a locking hole, and the locking hole extends along a longitudinal direction;
a body part, connected to the crown part and forming a fixing hole, the fixing hole extends along a transverse direction and communicates the locking hole; and
a holding part, connected to the body part and forming a holding groove and a through hole, the holding groove is suitable for clamping with at least a part of the fixed object, and the holding groove communicates to the fixing hole through the through hole;
a fixing piece, suitable to be penetratingly disposed at the fixing hole and comprising a fixing surface and an abutted surface, the fixing surface corresponds to at least a part of a surface of the fixed object, and the abutted surface is configured relative to the fixing surface; and
a locking piece, suitable to be penetratingly disposed at the locking hole and comprising an abutting surface, and the abutting surface optionally abuts against the abutted surface.

2. The strap fixing device according to claim 1, wherein the locking hole is a screw hole, and the locking piece is suitable to be screwed into the screw hole.

3. The strap fixing device according to claim 1, wherein the body part comprises at least one bearing surface, and the at least one bearing surface is configured to be adjacent to the fixing hole, the fixing piece further comprises at least one clamping surface, the at least one clamping surface is configured to be adjacent to the fixing surface and suitable to abut against the at least one bearing surface.

4. The strap fixing device according to claim 3, wherein the at least one bearing surface and the at least one clamping surface are plural, the plural bearing surfaces are symmetrically configured relative to the through hole, and the plural clamping surfaces are symmetrically configured relative to the fixing surface.

5. The strap fixing device according to claim 1, wherein the fixed object is a cylinder, and a cross-section of a groove wall of the holding groove substantially forms a major arc corresponding to a cross-section of the cylinder.
